(19) United States
(12) Patent Application Publication
(10) Pub. No.: US 2014/0303588 A1
(43) Pub. Date: Oct. 9, 2014

(54) DISPOSABLE DIAPER WITH ATTACHED WRAPPER FOR ENCLOSING AND SEALING THE DIAPER WHEN SOILED

(71) Applicant: DANIELLE TAVOLACCI, Mount Kisco, NY (US)

(72) Inventor: DANIELLE TAVOLACCI, Mount Kisco, NY (US)

(21) Appl. No.: 14/301,411

(22) Filed: Jun. 11, 2014

Related U.S. Application Data
(63) Continuation-in-part of application No. 13/332,581, filed on Dec. 21, 2011.
(60) Provisional application No. 61/515,378, filed on Aug. 5, 2011.

(51) Int. Cl. A61F 13/551

(52) U.S. Cl. CPC A61F 13/5512; A61F 2013/55125
USPC 604/385.13

(57) ABSTRACT

A disposable diaper is provided with a wrapper is the shape of a shower cap, enabling a user to wrap and seal the diaper when soiled. The wrapper is attached to either the front or back waistband region of the diaper. In one embodiment, flexible strands, secured to the wrapper and extending outward on opposite sides of its central opening, may be tied together to close the opening and fully seal the package or pouch when the soiled diaper is inserted. In another embodiment, a single tab secured to one side of the central opening is used to secure and seal the diaper within the pouch by adhering the table to the enter surface of the wrapper.

### CROSS-REFERENCE TO RELATED APPLICATION

This application is a continuation-in-part of U.S. patent application Ser. No. 13/332,581, filed Dec. 21, 2011, which, in torn, claims priority from Provisional Application No. 61/515,378 filed Aug. 5, 2011.

### BACKGROUND OF THE INVENTION

The present invention relates to an improvement in a disposable diaper which allows a user to wrap and seal the diaper when soiled.

More particularly, the present invention relates to a diaper with an attacked diaper wrapper, permitting the user to enclose and seal the diaper prior to disposal to avoid mess and odor.

Disposable diapers are well known in the art. Such diapers include a front waistband region, a back waistband region, and an intermediate region with two laterally opposed side margins interconnecting the front and back waistband regions. The intermediate region of the diaper has a liquid pervious liner, a liquid impervious backsheet and an absorbent core disposed between the liner and the backsheet.

When soiled by a wearer, the disposable diaper is normally disposed of by folding or rolling it into a packet which contains the liquid and/or solid waste end than dropping it into a liquid-impervious plastic trash bag. This disposal method is quite satisfactory if a trash container with a trash bag liner is close at hand. However, a diaper must often be changed, and the soiled diaper disposed of, while the wearer is in transit and such a trash bag is not readily available.

Various attempts have been made to design disposable diapers with an integrated means for wrapping and sealing the diaper when it is soiled. One such means is disclosed in the U.S. Pat. No. 4,923,455 entitled “Disposable Diaper with Integral Disposal Envelope”. This patent teaches attaching an “envelope” or bag-shaped container to one side of the diaper. When the diaper is soiled, it is folded and inserted inside the envelope. The edge of the envelope is provided with a seam that serves to close and seal the bag when the used diaper is inserted.

The U.S. Pat. No. 4,931,052 to Feldman discloses a disposable diaper having a “storage container” in the form of a plastic trash bag attached to its outer surface by “glue or fusion.”The trash bag is closeable, after inserting the soiled diaper therein, by means of a “drawstring” as is conventional with trash bags. Also attached to the diaper or to the storage container is a separate towelette for use and disposal with the soiled diaper prior to applying a new diaper to an infant.

The U.S. Pat. No. 4,674,135 to Greene discloses a “disposable undergarment” (panty) having a round “container portion” or pouch integrally formed on one side for use when disposing of the soiled garment. The pouch has elastic stitching around its upper periphery to draw the material together and form a stretchable elastic opening at the top.

Disposable diapers with these types of wrapping devices have not seen common commercial use because they add substantially to the cost of producing the diaper and are somewhat cumbersome to use.

### SUMMARY OF THE INVENTION

It is an object of the present invention to provide an inexpensive article, adapted to be used with a disposable diaper, which permits the diaper, when soiled, to be wrapped and sealed in a convenient way.

It is a further object of the present invention to provide a wrapper for a disposable diaper which enables the user to completely enclose and seal the diaper prior to disposal.

It is a further object of the present invention to provide a disposable diaper with an attached diaper wrapper that is inexpensive to produce, extremely easy to use and provides a sanitary way to dispose of the soiled diaper.

This object, as well as other objects which will become apparent from the discussion that follows, are achieved, in accordance with the present invention, by providing a wrapper in the shape of a “shower cap”, adapted to be attached to, and/or embedded in, either the front or back of a disposable diaper and having a closure device at the opening of the wrapper to allow the user to fully seal the package or pouch when the soiled diaper is inserted.

More particularly, the present invention provides a wrapper for a disposable diaper which comprises the following elements:
(a) a liquid impervious, disc-shaped, flexible sheet with a rounded enter periphery having a first width when laid flat; and
(b) an elastic strand secured to said outer periphery of the flexible sheet, the elastic strand forming an endless loop which, in its related state, shapes the flexible sheet into a pouch with an opening inside of said loop having a second width less than or substantially equal to a distance between said two side margins of said diaper.

The first width of the flexible sheet, which is preferably a plastic film, is at least twice the second width of the opening such that, when shaped into a pouch, the flexible sheet is caused to assume a round pocket or pouch shape having an outer, annular portion folded over with the opening at its center. The annular portion of the flexible sheet may be attached to one of the front and back waistband portions of the diaper or the entire flexible sheet may be embedded within the diaper itself between the absorbent intermediate layer and the liquid impervious backsheet.

According to a first preferred embodiment of the invention, the closure device comprises a pair of flexible strands secured to the flexible disc on opposite sides of the opening and extending away from each other in opposite directions. When the annular portion is attached to one of the front and back waistband portions of the diaper, the flexible strands extend outward substantially perpendicularly to the side margins of the diaper. With this arrangement, the diaper, when soiled, may be inserted in the pouch and the opening in the pouch may be closed by tying the flexible strands together, thereby wrapping and sealing the soiled diaper.

In this embodiment of the present invention, the annular portion of the flexible sheet may be attached to either the front or back waistband region of the diaper by means of an adhesive, by the application of heat and pressure, by sewing stitches or by any other means.

Alternatively, the wrapper may be enclosed in a wrapper cover that surrounds, protects and seals the wrapper. The enclosed wrapper may be sold as a separate commercial item or, preferably, the cover may be attached to one of the front and back waistband regions of the diaper.

The flexible strands used for closing the opening in the wrapper are preferably made of a liquid impervious material, such as plastic, and are folded around the elastic band and attached to both sides of the flexible sheet, for example, by means of an adhesive, by the application of heat and pressure or by sewing stitches.

It will be seen that the flexible sheet with the elastic band assumes a structure similar to that of a shower cap. In fact, a shower cap, when made in quantity by mechanical production equipment, is extremely inexpensive. Upon attaching suitable flexible strands to the opening of the shower cap, the cap may be simply attached to a disposable diaper in a final manufacturing step in producing the diaper.

According to a second preferred embodiment of the present Invention the closure device comprises a tab secured to the flexible sheet on one side of the opening and extending away from the opening. The tab is provided with means, such as a self-sticking, contact adhesive, for securing itself to the outer surface of the flexible sheet when the diaper is inside of said pouch. This embodiment provides a particularly effective way to seal the diaper within the pouch and avoid the escape of liquids and/or gasses (odor).

The self-sticking adhesive is preferably covered with a protective overlay sheet that can be removed prior to attaching the tab to the surface of the flexible sheet.

Preferably flexible sheet is disposed within said diaper itself, between its liner and its backsheet, in a position adjacent one edge of said absorbent core. The flexible sheet can be removable from said diaper through a slit in said backsheet.

In this case, flexible sheet is preferably attached to the liquid pervious layer of said diaper, in a position opposite to said slit in said backsheet, for example, by means of an adhesive, by the application of heat and pressure or by sewing stitches.

Preferably the tab is liquid impervious and may be made of plastic strip.

Alternatively, the means for securing the tab to the flexible sheet may include a hook and loop connector, with one part affixed to the tab and a mating strip affixed to the flexible sheet. The two parts are configured with hooks and loops respectively to adhere together and secure the tab to the surface of said flexible sheet when the diaper is disposed in said pouch.

For a full understanding of the present invention, reference should now be made to the following detailed description of the preferred embodiments of the invention as illustrated in the accompanying drawings.

### BRIEF DESCRIPTION OF THE DRAWINGS

FIG. 1 is a perspective view of the disposable diaper with an attached wrapper according to a first preferred embodiment of the present invention.

FIG. 1a is a cross-sectional view of an intermediate region of a diaper.

FIG. 2 is a top view of the disposable diaper of FIG. 1 with the attached wrapper shown in dashed lines.

FIG. 3 is a side view of a typical shower cap which may be used with the present invention.

FIG. 4 is a top view of a shower cap with attached strands extending outward in opposite directions, for wrapping and sealing a soiled diaper in accordance with the first preferred embodiment of the present invention.

FIG. 5 is a representational, cross-sectional diagram showing a shower cap and the attached strands of FIG. 4 in cross section, with the strands wrapped around the edge of the shower cap at the opening.

FIG. 6 is a representational, cross-sectional diagram showing a disposable diaper with an attached wrapper according to the first preferred embodiment of the present invention.

FIG. 6A is a representational, cross-sectional diagram showing an alternative embodiment of the disposable diaper with the wrapper enclosed in a protective cover which, in term, is attached to the diaper.

FIG. 7 is a representational diagram showing the final step in the manufacture of a disposable diaper and attached wrapper according to the first preferred embodiment of the present invention.

FIG. 8 is a perspective view of closed and sealed wrapper forming a pouch containing a soiled disposable diaper according to the first preferred embodiment of the invention

FIGS. 9A and 9B are cross-sectional and representational diagrams showing a disposable diaper with an attached wrapper according to a second preferred embodiment of the present invention.

FIGS. 10A and 10B are illustrations of the diaper wrapper of FIG. 9 with its associated diaper inserted in the pouch (FIG. 10A) and wrapped tightly (FIG. 10B).

FIG. 11 is a cross-sectional and representational diagram of the diaper wrapper of FIGS. 9 and 10 showing the tab used to close the pouch.

### DESCRIPTION OF THE PREFERRED EMBODIMENTS

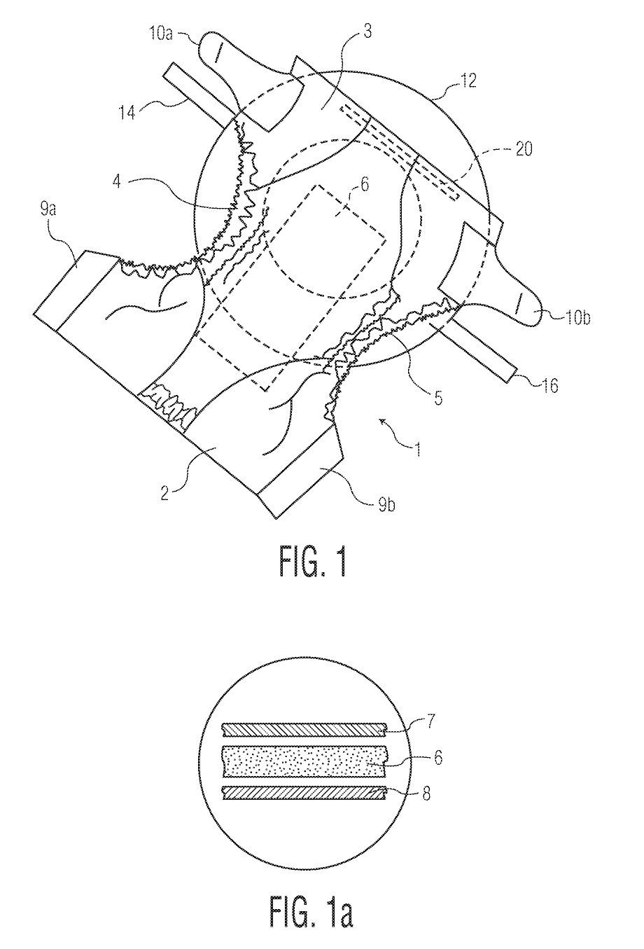

The preferred embodiments of the present invention will now be described with reference to FIGS. 1-11 of the drawings. Identical elements in the various figures are designated with the same reference numerals.

FIG. 1 shows a disposable diaper with an attached wrapper according to a first preferred embodiment of the present invention for enclosing and sealing the diaper when soiled. The disposable absorbent diaper 1 has a front waistband region 2, a back waistband region 3 and an intermediate region with two laterally opposed side margins 4 and 5, respectively, interconnecting the front and back waistband regions. As shown in FIG. 1a, the intermediate region of the diaper has a liquid pervious liner 7, a liquid impervious backsheet 8 and an absorbent core 6 interposed between the liner and the backsheet.

The front waistband region 2 has tabs 9a and 9b on opposite sides. The back waistband region 3 has matching tabs 10a and 10b on opposite sides. The tabs 9a and 10a are attachable together and the tabs 9b and 10b are attachable together to retain the diaper on an infant.

According to one embodiment of the present invention, the diaper farther includes a wrapper 12 with attached, oppositely directed flexible strands 14 and 16 for enclosing and sealing the diaper when soiled.

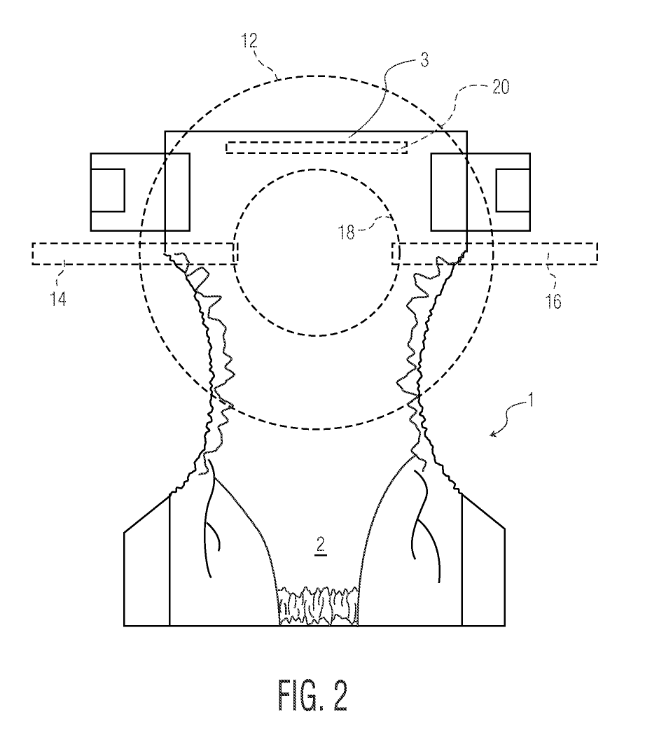

As is best seen in FIG. 2, which is a plan view of the diaper 1 with the wrapper 12 indicated in dashed lines, and FIG. 4 which shows the wrapper 12 alone, the wrapper has an opening 18 at the center for receiving the soiled diaper. The wrapper 12 may be attached to either the front or back waistband of the diaper, as indicated by the dashed lines 20 in FIG. 2, by means of an adhesive, by the application of heat and pressure and/or by sewing stitches.

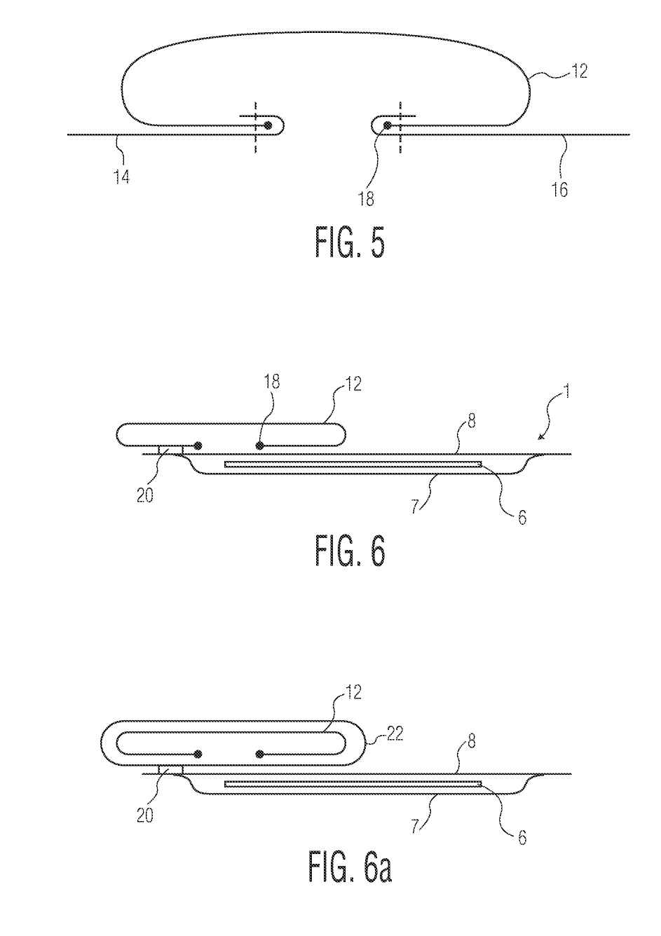

The flexible strands 14 and 16 are preferably ribbon shaped and liquid impervious. They are preferably made of durable plastic sheet, for example. As shown in FIG. 5, the strands are preferably folded around the inner edge of the opening 18 of the wrapper 12. They can be secured to the wrapper 12 by an adhesive, by heat and pressure and/or by sewing stitches.

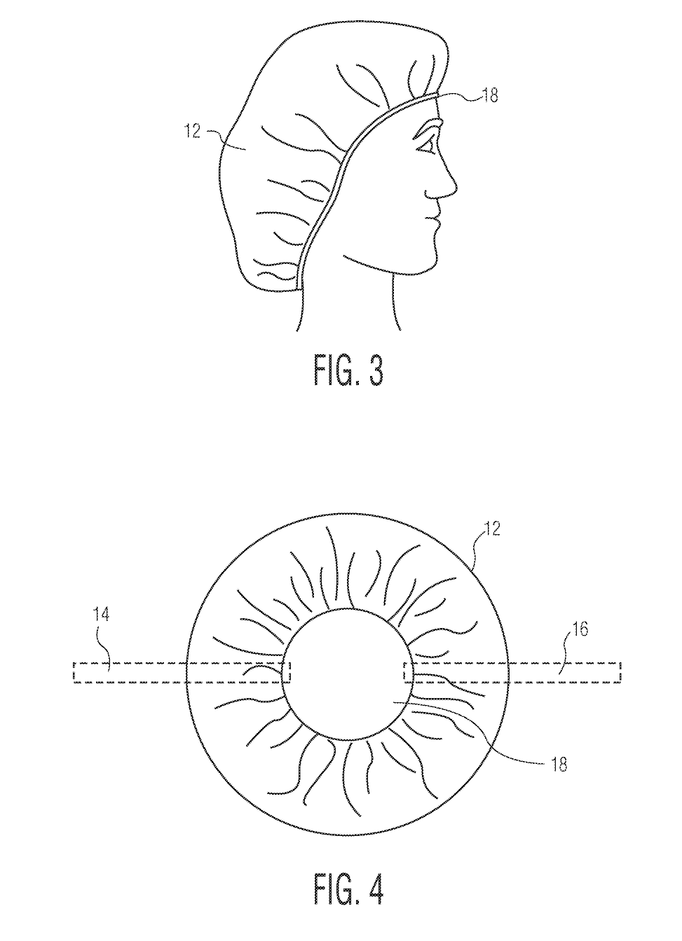

In this preferred embodiment of the present invention, the wrapper 12 takes the form of a conventional shower cap as shown in FIG. 3. This cap is formed by a liquid impervious, circular or oval shaped flexible sheet. An elastic strand is secured to the periphery of the opening 18 for the head, forming an endless loop which, in its relaxed state, shapes the flexible sheet into a pouch with the opening inside of the loop. The width of this opening 18 is preferably less than, or substantially equal to, the distance between the two side margins 4 and 5 of the diaper to be wrapped.

Preferably, the width of the flexible sheet when laid flat, without the elastic strand, is at least twice the width of the opening, such that, when shaped into a poach as shown in FIG. 4, the flexible sheet is thereby caused to assume a rounded pocket or pouch shape having an enter, annular portion folded over with the opening 18 at the middle.

Since shower caps, which are either substantially circular or oval shaped when laid flat, are produced in great quantities, they provide an inexpensive starting point for producing a wrapper for a disposable diaper. It is necessary only to attach the strands 14 and 16 and, thereafter, to attach the wrapper to a disposable diaper at the end of the diaper manufacturing process.

The wrapper can also he an individual article of commerce, sold separately from a disposable diaper and used for the same purpose of enclosing and sealing a diaper.

FIG. 6 illustrates, in cross-section, how the wrapper 12 is attached to the impervious backsheet 8 of a diaper 1 which, as shown, includes a pervious layer 7 and an absorbent layer 6. The attachment 20 is by any conventional means, such as by an adhesive, by the application of heat and pressure, and/or by sewing stitches.

FIG. 6A illustrates an alternative embodiment wherein the wrapper 12 is enclosed in a protective cover 22 which, in turn, is attached to the diaper 1 by a attachment means 20. The protective cover is preferably made of a liquid impervious material.

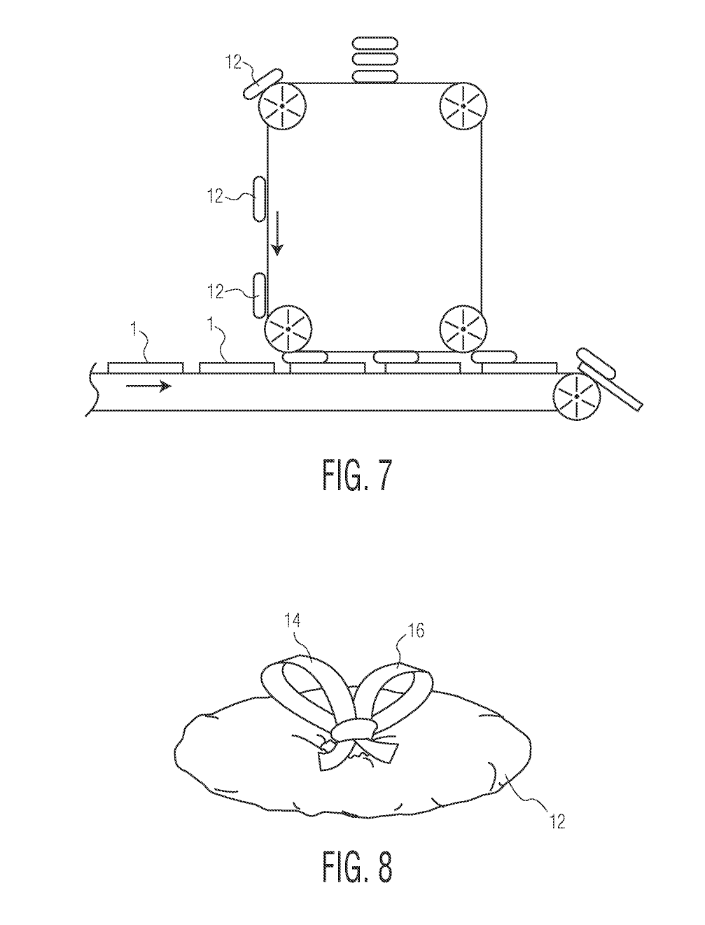

The wrapper 12 can be attached to the diaper 1 in a final manufacturing step, as indicated by the dual conveyor system shown in FIG. 7.

FIG. 8 shows a wrapper 12 with its opening 13 closed by the strands 14 and 16, thereby sealing its contents: the soiled diaper. When closed and sealed in this way, there is neither mess nor odor and the package can be retained for later disposal.

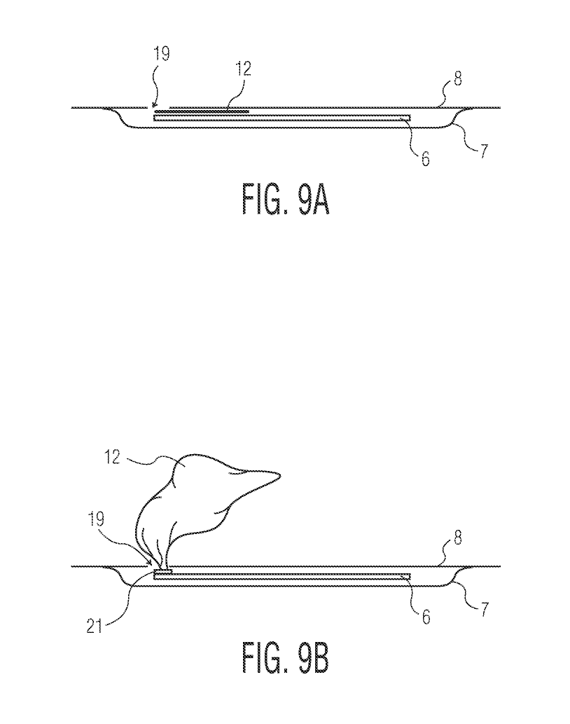

FIGS. 9-11 illustrate a second preferred embodiment of the invention in which the diaper wrapper 12 is provided with a single tab 24, instead of the two oppositely directed strands 14 and 16, for closing the pouch with the soiled diaper inside.

As seen in FIG. 9A, the diaper wrapper 12 is initially disposed within the diaper itself. In particular, the wrapper is sandwiched between the liquid pervious layer 7 and the liquid impervious backsheet 8, near one edge of the central absorbent core layer 6.

As is illustrated in FIG. 9B, the wrapper 12 is removable from the diaper through a slit 19 in the backsheet 18. The diaper wrapper is preferably attached to either the core layer 6 or the pervious layer 7, as shown at 21, to prevent its complete removal.

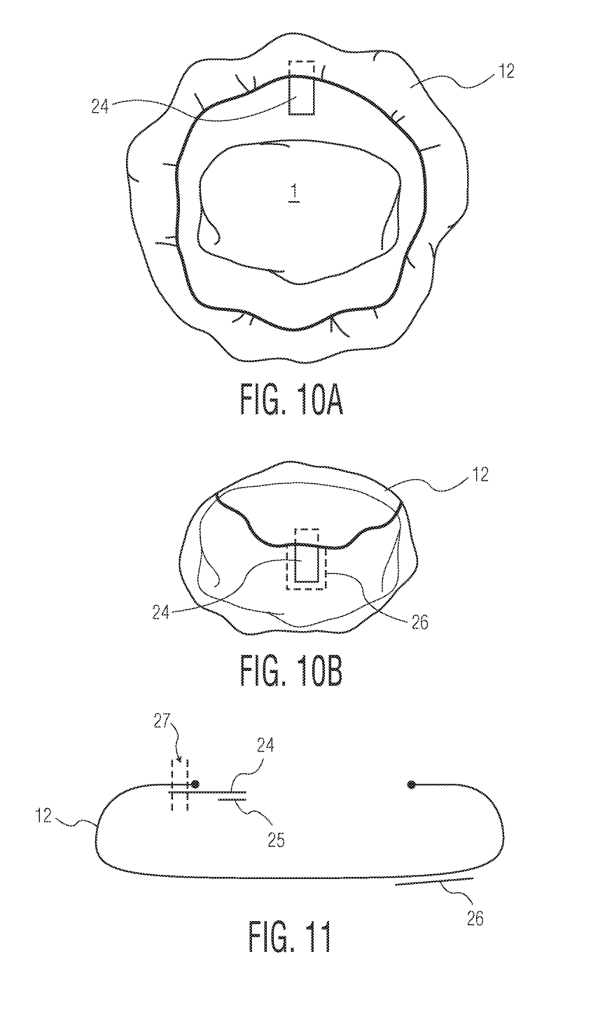

FIGS. 10A and 10B show how the diaper is wrapped, once the diaper wrapper 12 is removed therefrom in the manner illustrated in FIG. 9B. The diaper 1 is folded and placed in the pouch formed by the wrapper 12 and then secured inside, to prevent the escape of liquids and/or gases, by means of a tab 24.

The tab can be secured directly to the outer surface of the diaper wrapper 12 by means of a contact adhesive, or by a hook and loop fastener (Velcro®). FIG. 11 shows the tab 24 attached to an edge of the wrapper 12 by an adhesive, by the application of heat and pressure or by sewing, in the area indicated by the dashed lines 27. The tab 24 is preferably provided with a self-sticking contact adhesive, covered by a removable overlay 25, for attachment to the outer surface of the flexible sheet, or it may be provided with hook and loop layers 25 and 26, respectively, that adhere together upon contact.

There has thus been shown and described a novel disposable diaper with attached wrapper for enclosing and sealing the diaper when soiled which fulfills all the objects and advantages sought therefor. Many changes, modifications, variations and other uses and applications of the subject invention will, however, become apparent to those skilled in the art after considering this specification and the accompanying drawings which disclose the preferred embodiments thereof. All such changes, modifications, variations and other uses and applications which do not depart from the spirit and scope of the invention are deemed to be covered by the invention, which is to foe limited only by the claims which follow.

What is claimed is:

1. A disposable absorbent diaper including a front waistband region, a back waistband region, and an intermediate region with two laterally opposed side margins interconnecting the front and back waistband regions, the intermediate region of the diaper having a liquid pervious liner, a liquid impervious backsheet and an absorbent core disposed between the liner and the backsheet, said diaper further including a wrapper for enclosing and sealing the diaper when soiled, with said wrapper comprising:
(a) a liquid impervious, disc-shaped, flexible sheet with a rounded outer periphery having a first width when laid flat;
(b) an elastic strand sectored to said outer periphery of the flexible sheet, said elastic strand forming an endless loop which, in a relaxed state thereof, shapes the flexible sheet into a pouch with an opening inside of said loop having a second width less than or substantially equal to a distance between said two side margins of said diaper, wherein said first width of the flexible sheet is at least twice said second width of said opening such that, when shaped into a pouch, said flexible sheet is thereby caused to assume a rounded pouch shape having an outer, annular portion folded over with said opening at a center portion thereof; and
(c) a tab secured to the flexible sheet on one side of said opening and extending away from said opening, said tab having adhering means for securing itself to a surface of the flexible sheet when the diaper is inside of said pouch;
whereby said diaper, when soiled, may be inserted in said pouch and the opening in said pouch may be closed by means of said tab, thereby wrapping and sealing the soiled diaper within said pouch.

2. The diaper defined in claim 1, wherein said flexible sheet is disposed within said diaper between said liner and said backsheet in a position adjacent one edge of said absorbent core, and said flexible sheet is removable from said diaper through a slit in said backsheet.

3. The diaper defined in claim 2, wherein said flexible sheet is attached to said liquid pervious layer of said diaper in a position opposite to said slit in said backsheet.

4. The diaper defined in claim 3, wherein said flexible sheet is attached to the liquid pervious layer by at least one of an adhesive, the application of heat and pressure, and by sewing stitches.

5. The diaper defined in claim 1, wherein said tab is liquid impervious.

6. The diaper defined in claim 1, wherein said tab is made of plastic.

7. The diaper defined in claim 1, wherein said securing means of said tab includes a self-adhering, contact adhesive.

8. The diaper defined in claim 7, wherein the tab includes a removable cover sheet overlaid said adhesive which, when removed, exposes the adhesive and allows said tab to be secured to the surface of said flexible sheet when said diaper is disposed in said pouch.

9. The diaper defined in claim 1, wherein said securing means includes (1) a hook and loop connector affixed to said tab and (2) a mating hook and loop connector strip affixed to said flexible sheet, said connectors being configured to adhere together and secure said tab to the surface of said flexible sheet when said diaper is disposed in said pouch.

10. The diaper defined in claim 1, wherein said tab is secured to said flexible sheet by an adhesive.

11. The diaper defined in claim 1, wherein said tab is secured to said flexible sheet by application of heat and pressure.

12. The diaper defined in claim 1, wherein said tab is secured to said flexible sheet by sewing stitches.

13. The diaper defined in claim 1, wherein said flexible sheet is substantially circular when laid flat.

14. The diaper defined in claim 1, wherein said flexible sheet is oval-shaped when laid flat.

15. The diaper defined in claim 1, wherein said flexible sheet is formed as a shower cap.
